(19) United States
(12) Patent Application Publication
(10) Pub. No.: US 2026/0071193 A1
(43) Pub. Date: Mar. 12, 2026

(54) EPSPS ENZYME MUTANT, NUCLEIC ACID MOLECULE, EXPRESSION CASSETTE, EXPRESSION VECTOR, RECOMBINANT BACTERIUM OR RECOMBINANT CELL AND USE THEREOF

(71) Applicant: GEVOTO LLC, Chengdu City (CN)

(72) Inventors: Yalan Tan, Chengdu City (CN); Qingjiang Hou, Chengdu City (CN); Yena Wei, Chengdu City (CN); Jiangbo Hu, Chengdu City (CN); Zhilin Zhang, Chengdu City (CN); Rong Chen, Chengdu City (CN); Yanan Xiong, Chengdu City (CN); Shunhua Sun, Chengdu City (CN); Yifan Yang, Chengdu City (CN); Xianglan Shen, Chengdu City (CN); Yang Feng, Chengdu City (CN); Xiaorong Feng, Chengdu City (CN); Nanfei Xu, Chengdu City (CN)

(21) Appl. No.: 19/322,887

(22) Filed: Sep. 9, 2025

(30) Foreign Application Priority Data
Sep. 9, 2024 (CN) 202411257192.4

(51) Int. Cl. C12N 9/10; C12N 15/82

(52) U.S. Cl. CPC C12N 9/1092; C12N 15/8205; C12N 15/8207; C12N 15/8275; C12Y 205/01019

(57) ABSTRACT

Provided are an EPSPS enzyme mutant, a nucleic acid molecule, an expression cassette, an expression vector, a recombinant bacterium or a recombinant cell and use thereof. The EPSPS enzyme mutant can effectively solve the problem of non-selectable damage to crops caused by herbicides such as glyphosate, and provides a new route for cultivating glyphosate-resistant crop varieties. Moreover, the EPSPS enzyme mutant also has relatively high practicability and promotional value, can improve a weed control effect, reduce weeding costs, and elevate crop yields in agricultural production. Besides, it further provides an expression cassette expressing the EPSPS enzyme mutant, where the expression cassette contains an EPSPS mutant gene conferring glyphosate resistance on a variety of plants such as soybean and rice.

### CROSS-REFERENCE TO RELATED APPLICATION

The present disclosure claims the priority to the Chinese patent application with the filling No. 202411257192.4 filed with the Chinese Patent Office on Sep. 9, 2024, and entitled “EPSPS ENZYME MUTANT, NUCLEIC ACID MOLECULE, EXPRESSION CASSETTE, EXPRESSION VECTOR, RECOMBINANT BACTERIUM OR RECOMBINANT CELL AND USE THEREOF”, the contents of which are incorporated herein by reference in entirety.

### REFERENCE TO SEQUENCE LISTING

This application includes a Sequence Listing filed electronically as an XML file named “ONI25302457US_Sequence Listing.xml”, created on Sep. 9, 2025, with a size of 51,596 bytes. The Sequence Listing is incorporated herein by reference.

### TECHNICAL FIELD

The present disclosure relates to the technical field of plant genetic engineering, and specifically to an EPSPS enzyme mutant, a nucleic acid molecule, an expression cassette, an expression vector, a recombinant bacterium or a recombinant cell and use thereof.

### BACKGROUND ART

Glyphosate is herbicide targeting 5-enolpyruvylshikimate-3-phosphate synthase (EPSPS) in plant chloroplasts. EPSPS is an enzyme involved in a shikimic acid biosynthetic pathway, and involved in synthesis of aromatic amino acids. Weed control mechanism of glyphosate is as follows. Glyphosate primarily achieves a weeding goal by disrupting a shikimic acid synthesis pathway of plants, and an action mechanism thereof primarily involves competitive inhibition of EPSPS activity in the shikimic acid pathway, thus preventing affected plants from continuously synthesizing essential aromatic amino acids that impair normal plant growth and even lead to death. However, as a non-selective herbicide, glyphosate efficiently controls weeds but simultaneously damages crops without discrimination. Therefore, cultivating and planting glyphosate-resistant crop varieties can not only reduce the burden of manual weeding but also increase crop yields, facilitating mechanized farming practices.

The glyphosate-resistant CP4 gene currently most commonly used in the market is a gene which is isolated by Monsanto company from Agrobacterium and has strong resistance to glyphosate. Crops with glyphosate resistance trait obtained by introducing CP4 gene through transgenic technology have been widely applied, and CP4-containing corn and soybean varieties have been widely popularized in the last two decades. However, transgenic crops have not been widely used for a variety of reasons. Therefore, discovery of novel plant-derived glyphosate-resistant genes and their use in crops have significant economic and commercial value.

In view of this, the present disclosure is specifically proposed.

### SUMMARY

Objective of the present disclosure lies in providing an EPSPS enzyme mutant, a nucleic acid molecule, an expression cassette, an expression vector, a recombinant bacterium or a recombinant cell and use thereof, so as to enable plants to be resistant to glyphosate, without affecting original functions of bio-enzymes. The inventors have further developed use of the mutein, coding gene, expression cassette and expression vector in transgenosis, gene editing or other plant breeding, which enable cultivation of plants, particularly crop plants, with resistance to glyphosate herbicides.

The present disclosure is implemented as follows.

In the first aspect, the present disclosure provides an EPSPS enzyme mutant, which is resistant to glyphosate, as shown in (1) or (2) below:
(1) obtained by mutating amino acids at sites 131, 172, 177, 274, 313 and 403 of a rice-derived wild-type EPSPS enzyme, where an amino acid sequence of the wild-type EPSPS enzyme is as set forth in SEQ ID NO: 2; and
amino acids at sites 131, 172, 177, 274, 313 and 403 of the EPSPS enzyme mutant are G, A, S, R, E and A, respectively, and an amino acid sequence thereof is as set forth in SEQ ID NO: 4; and
(2) having at least no less than 70% identity to the EPSPS enzyme mutant as shown in (1), having identical amino acids at sites 131, 172, 177, 274, 313 and 403 of the EPSPS enzyme mutant shown in (1), and being resistant to glyphosate.

For example, it has at least 70%, 71%, 72%, 73%, 74%, 75%, 76%, 77%, 78%, 79%, 80%, 85%, 90%, 91%, 92%, 93%, 94%, 95%, 96%, 97%, 98%, 99% or 100% identity to the EPSPS enzyme mutant as shown in (1).

The inventors found that, by performing the mutation at the above sites of the wild-type rice EPSPS enzyme into corresponding amino acids, the glyphosate-resistant EPSPS enzyme can be obtained, and after it is transformed into a plant, the plant can express the EPSPS enzyme mutant and has glyphosate resistance. The EPSPS enzyme mutant provided by the present disclosure can effectively solve the problem of non-selectable damage to crops caused by herbicides such as glyphosate, and provides a new route for cultivating glyphosate-resistant crop varieties. Moreover, the EPSPS enzyme mutant provided by the present disclosure also has relatively high practicability and promotional value, can be used for breeding a variety of glyphosate-resistant crops such as rice and soybean, and can improve a weed control effect, reduce weeding costs, and elevate crop yields in agricultural production.

Expression of the above EPSPS enzyme mutant in soybeans can confer tolerance to herbicides inhibiting the EPSPS enzyme, particularly herbicides of the phosphonomethylglycine family, including but not limited to glyphosate, on soybeans.

As defined herein, “glyphosate” includes any herbicidally effective form of N-phosphonomethylglycine (including any salt thereof), other forms that generate glyphosate anions in plants, and any other herbicides within the phosphonomethylglycine family.

The EPSPS enzyme in the present disclosure refers to 5-enolpyruvylshikimate-3-phosphate synthase.

Plants (such as rice (Oryza sativa) or soybean (Glycine max)) transformed with the above EPSPS mutation have higher glyphosate tolerance than the same plant species transformed with the glyphosate-resistant genes, i.e., CP4-EPSPS gene and GOX gene. GOX refers to a glyphosate oxidoreductase gene.

In the second aspect, the present disclosure provides a nucleic acid molecule encoding the above EPSPS enzyme mutant.

The term “nucleic acid molecule encoding the EPSPS enzyme mutant” may be a polynucleotide encoding the mutein of the present disclosure, or further may be a polynucleotide containing an additional coding and/or non-coding sequence.

In cases where the present disclosure provides the above amino acid sequence, those skilled in the art would have readily obtained the nucleic acid sequence encoding the above EPSPS enzyme mutant according to the degeneracy of codons. For example, a nucleic acid sequence encoding the above EPSPS enzyme mutant can be obtained by mutating corresponding nucleotides on the nucleic acid sequence encoding the wild-type EPSPS enzyme. This would be readily achievable to those skilled in the art.

The mutein and polynucleotide of the present disclosure are preferably provided in an isolated form, and more preferably purified to homogeneity.

A polynucleotide full-length sequence of the present disclosure generally can be obtained by a PCR amplification method, a recombinant method or an artificial synthesis method. For the PCR amplification method, primers can be designed based on a relevant nucleotide sequence disclosed in the present disclosure, particularly an open reading frame sequence, and relevant sequences are obtained by amplification using a commercially available cDNA library or a cDNA library prepared by a conventional method known to those skilled in the art as a template. When a sequence is relatively long, two or more PCR amplifications often need to be performed before various amplified fragments are assembled in a correct order.

Once the relevant sequence is obtained, relevant sequences can be obtained in large quantities by the recombinant method, typically involving cloning it into a vector, transferring into a cell, and isolating relevant sequences from a host cell after propagation by a conventional method.

In addition, relevant sequences also can be synthesized by the artificial synthesis method, particularly when fragments are relatively short. Generally, a fragment with a very long sequence can be obtained by first synthesizing a plurality of small fragments, and then linking them.

At present, it is entirely feasible to obtain an DNA sequence encoding the protein (or fragments or derivatives thereof) of the present disclosure through chemical synthesis. The DNA sequence then can be introduced into various existing DNA molecules (or, e.g., vectors) and cells known in the art. Moreover, mutation also can be introduced into a protein sequence of the present disclosure through chemical synthesis.

A method of applying PCR technology to amplify DNA/RNA is preferably employed to obtain the polynucleotide of the present disclosure. Particularly when it is difficult to obtain full-length cDNA from a library, the RACE method (RACE-cDNA, rapid amplification of cDNA ends) may be preferably utilized. Primers for PCR can be appropriately selected based on sequence information of the present disclosure disclosed herein, and can be synthesized by a conventional method. An amplified DNA/RNA fragment can be isolated and purified by a conventional method such as gel electrophoresis.

In a preferred embodiment of applying the present disclosure, the nucleotide sequence of the nucleic acid molecule is as set forth in SEQ ID NO: 3. In order to improve translational efficiency of the coding gene of the EPSPS enzyme mutant in soybeans, the present disclosure optimizes the nucleotide sequence based on the codon bias of soybean nucleotide sequence, as set forth in SEQ ID NO: 3. The nucleotide sequence of the original wild-type rice EPSPS is as set forth in SEQ ID NO:1, and the sequence as set forth in SEQ ID NO: 3 has 77% homology to the nucleotide of the original wild-type rice EPSPS.

In the third aspect, the present disclosure provides a nucleic acid molecule, including an intron and an exon sequence encoding the above EPSPS enzyme mutant.

In a preferred embodiment of applying the present disclosure, the nucleic acid molecule includes: sequences of a first exon, a first intron, a second exon, a second intron, a third exon, a third intron, a fourth exon, a fourth intron, a fifth exon, a fifth intron, a sixth exon, a sixth intron, a seventh exon, a seventh intron and an eighth exon arranged successively in a 5′-3′ direction, where sequences are as set forth in SEQ ID NOs: 10-24, respectively.

The introns include, but are not limited to, introns of Adh1, bronze 1, actin 1, actin 2 or sucrose synthase introns.

In a preferred embodiment of applying the present disclosure, a sequence of the nucleic acid molecule is as set forth in SEQ ID NO: 5.

The nucleotide sequence as set forth in SEQ ID NO: 5 is a full-length gene sequence (including intron sequences and exon sequences) of the EPSPS enzyme mutant.

In an optional embodiment, the nucleic acid molecule is selected from DNA, RNA, or a combination thereof.

In the fourth aspect, the present disclosure provides an expression cassette, including the above nucleic acid molecule.

In a preferred embodiment of applying the present disclosure, the expression cassette further includes a promoter and a terminator.

When the nucleic acid molecule is a nucleic acid molecule that does not include introns but only exons, in a preferred embodiment of applying the present disclosure, the promoter includes, but is not limited to: any one selected from the group consisting of a cauliflower mosaic virus 19S promoter, a cauliflower mosaic virus 35S promoter, a figwort mosaic virus 35S promoter, a sugarcane baculovirus promoter, a commelina yellow mottle virus promoter, a ribulose-1,5-bisphosphate carboxylase (rubisco) small subunit promoter, a cassava vein mosaic virus (CsVMV) promoter, an arabidopsis ubiquitin 10 (UBI10) gene promoter, a rice actin1 promoter, a mannopine synthase gene promoter, an octopine synthase gene promoter, a soybean ubiquitin (GmUBI) gene promoter, a maize ubiquitin promoter and a rice EPSPS gene promoter.

When the nucleic acid molecule is a nucleic acid molecule that does not include introns but only exons, in a preferred embodiment of applying the present disclosure, the terminator includes, but is not limited to: any one selected from the group consisting of a terminator of nopaline synthase gene (NOS) from Agrobacterium tumefaciens, an octopine synthase gene terminator, a terminator of 19S gene of CaMV, a terminator of 35S gene of CaMV, a mannopine synthase gene terminator, a terminator of alcohol dehydrogenase tAdh gene from Saccharomyces cerevisiae, a terminator of trpC gene from Aspergillus nidulans and a terminator of rice EPSPS gene.

In a preferred embodiment of applying the present disclosure, when the nucleic acid molecule of the expression cassette is a nucleic acid molecule including introns, a sequence of the promoter of the expression cassette is as set forth in SEQ ID NO: 6, and SEQ ID NO: 6 sets forth a promoter of the rice EPSPS gene.

In a preferred embodiment of applying the present disclosure, when the nucleic acid molecule of the expression cassette is a nucleic acid molecule including introns, a sequence of the terminator of the expression cassette is as set forth in SEQ ID NO: 7, and SEQ ID NO: 7 sets forth a terminator of the rice EPSPS gene.

When the above promoter and terminator are selected from the promoter and terminator of the rice EPSPS gene, as the expression cassette is derived from rice varieties itself rather than from microorganisms, it is applicable to transform a variety of plant varieties, such as rice, tobacco, soybean, maize, cotton, sorghum, and wheat, and it has a broader application range.

In an embodiment, the expression cassette contains a promoter, an intron and a terminator, where the intron includes, but is not limited to, an intron enhancing transgene expression, such as artificial and natural introns, e.g., RBG intron.

In a preferred embodiment of applying the present disclosure, a sequence of the expression cassette is as set forth in SEQ ID NO: 8.

In the fifth aspect, the present disclosure provides an expression vector, including the above expression cassette.

### Expression Vector

The present disclosure further relates to a vector containing the nucleic acid molecule of the present disclosure, a host cell produced genetically engineered with the vector of the present disclosure or the mutant protein coding sequence of the present disclosure, and a method for generating the EPSPS enzyme mutant of the present disclosure by recombination technique.

In the present disclosure, a polynucleotide sequence encoding the mutein can be inserted into a recombinant expression vector. The term “recombinant expression vector” refers to bacterial plasmids, bacteriophages, yeast plasmids, plant cell viruses, mammalian cell viruses, such as adenoviruses and retroviruses, or other vectors well-known in the art. Any plasmid and vector can be used as long as it is capable of replication and stabilization in the host. One important characteristic of the expression vector is typically containing an origin of replication, a promoter, a marker gene and a translational control element.

In the sixth aspect, the present disclosure provides a recombinant bacterium or a recombinant cell, where the recombinant bacterium or the recombinant cell contains a coding gene encoding the above EPSPS enzyme mutant, and the recombinant cell is a non-plant cell.

In a preferred embodiment of applying the present disclosure, the recombinant bacterium or the recombinant cell is a bacterium or a fungus.

In a preferred embodiment of applying the present disclosure, the bacterium is Agrobacterium or E. coli; and the fungus is yeast. The recombinant cell may be a competent cell.

The recombinant cell may be a prokaryotic cell, such as a bacterial cell, or a lower eukaryotic cell, such as yeast cell, or a higher non-plant eukaryotic cell, such as mammalian cell. Representative examples are: bacterial cells of E. coli, Streptomyces, and Salmonella typhimurium; and fungal cells, such as yeast.

In the seventh aspect, the present disclosure provides use of the EPSPS enzyme mutant, the nucleic acid molecule, the expression cassette, the expression vector or the recombinant bacterium or recombinant cell in cultivating a glyphosate-resistant plant.

In a preferred embodiment of applying the present disclosure, it includes at least one of following use modes:
(1) delivering the nucleic acid molecule into a cell of a plant of interest;
(2) introducing the expression cassette into a plant of interest by a particle bombardment or an Agrobacterium-mediated transformation method, where the expression cassette includes, but is not limited to, an EPSPS promoter, an intron, an exon encoding an enzyme of SEQ ID NO: 4 and an EPSPS terminator;
(3) transforming a plant of interest with the vector, where the vector contains a coding gene encoding a glyphosate-resistant rice EPSPS enzyme mutant;
(4) introducing the recombinant bacterium or the recombinant cell into a plant of interest, where the recombinant bacterium or the recombinant cell contains a coding gene encoding a glyphosate-resistant rice EPSPS enzyme mutant; and
(5) performing gene editing on a cell of a plant of interest, to allow it to encode the above EPSPS enzyme mutant.

In an optional embodiment, an introduction method is selected from a genetic transformation method, a genome editing method or a gene mutation method.

The above genetic transformation method includes, but is not limited to, producing glyphosate-resistant individuals by inbreeding parent plants carrying a gene of the glyphosate-resistant EPSPS enzyme mutant, or by hybridizing with other plant individuals.

In other embodiments, the above transformation method includes, but is not limited to, an Agrobacterium-mediated gene transformation method, a gene-gun transformation method, and a pollen tube pathway method.

In a preferred embodiment of applying the present disclosure, the plant is selected from the group consisting of rice, tobacco, soybean, maize, cotton, sorghum, wheat and oilseed rape.

In a preferred embodiment of applying the present disclosure, the plant is rice or soybean.

In the eighth aspect, the present disclosure provides a method for cultivating a glyphosate-resistant plant, where an EPSPS enzyme in a cell of a plant of interest and a rice-derived wild-type EPSPS enzyme are subjected to sequence alignment, so as to enable mutation of amino acids of the EPSPS enzyme of the cell of the plant of interest corresponding to sites 131, 172, 177, 274, 313 and 403 of the wild-type rice EPSPS enzyme into G, A, S, R, E and A, respectively, where an amino acid sequence of the wild-type EPSPS enzyme is as set forth in SEQ ID NO: 2.

The above method for cultivating the glyphosate-resistant plant includes, but is not limited to, performing sequence alignment on the EPSPS enzyme in the cell of the plant of interest and the wild-type rice EPSPS enzyme that have any homology, so as to enable mutation of the amino acids of the EPSPS enzyme of the cell of the plant of interest corresponding to the sites 131, 172, 177, 274, 313 and 403 of the wild-type rice EPSPS enzyme into G, A, S, R, E and A, respectively, so that the glyphosate-resistant plant can be screened and cultivated.

Mutation methods include, but are not limited to, gene editing, where gene editing is performed on a coding gene in the cell of the plant of interest, so as to allow adaptive mutation to the amino acids at corresponding sites in the above.

In a preferred embodiment of applying the present disclosure, the plant of interest is selected from the group consisting of rice, tobacco, soybean, maize, cotton, sorghum, wheat and oilseed rape.

In the ninth aspect, the present disclosure provides a method for detecting an EPSPS mutant plant resistant to glyphosate, including: determining whether a plant to be tested contains the above nucleic acid molecule; or determining whether the plant to be tested contains the above EPSPS enzyme mutant.

Determining whether rice to be tested contains the above nucleic acid molecule includes, but not limited to, methods such as using detection primers, probes, aptamers, and chips for determination. Detection of the nucleic acid molecule is realized by amplification and/or hybridization, etc.

A method for determining whether the rice to be tested contains the above EPSPS enzyme mutant resistant to glyphosate includes, but is not limited to, modifying a marker capable of detecting the EPSPS enzyme mutant on a solid support so as to realize qualitative or quantitative determination of the EPSPS enzyme mutant. The solid support includes, but is not limited to, magnetic bead, test strip, microtiter plate, membrane, micron-sized particle, nanoparticle, etc.

The present disclosure has following beneficial effects.

In the present disclosure, by performing the mutation at the above sites of the wild-type rice EPSPS enzyme, the glyphosate-resistant EPSPS enzyme can be obtained, and after it is transformed into a plant, the plant can express the EPSPS enzyme mutant and is resistant to glyphosate. The EPSPS enzyme mutant provided by the present disclosure can effectively solve the problem of non-selectable damage to crops caused by herbicides such as glyphosate, and provides a new route for cultivating glyphosate-resistant crop varieties. Moreover, the EPSPS enzyme mutant provided by the present disclosure also has relatively high practicability and promotional value, and can improve the weed control effect, reduce weeding costs, and elevate crop yields in agricultural production.

The present disclosure provides expression of the EPSPS enzyme mutant in plants such as soybean and rice, and can confer tolerance to herbicides inhibiting the EPSPS enzyme, particularly herbicides of the phosphonomethylglycine family, including but not limited to glyphosate, on the plants such as soybean and rice.

Upon transformation of the EPSPS mutation with A131G, G172A, P177S, K274R, K313E and V403A into a plant, the plant (such as rice or soybean) has higher glyphosate tolerance than the same plant species transformed with the glyphosate-resistant genes, i.e., CP4-EPSPS gene and GOX gene.

In addition, the present disclosure further provides an expression cassette for expressing an EPSPS enzyme mutant, where the expression cassette contains an EPSPS mutant gene conferring glyphosate resistance on a variety of plants such as soybean and rice.

The EPSPS enzyme mutant, nucleic acid molecule, expression cassette, vector, recombinant bacterium or recombinant cell resistant to glyphosate provided by the present disclosure have broad application prospects in the cultivation of glyphosate-resistant soybean.

### BRIEF DESCRIPTION OF THE DRAWINGS

In order to illustrate technical solutions of embodiments of the present disclosure more clearly, drawings which need to be used in the embodiments will be briefly introduced below. It should be understood that the drawings merely show some embodiments of the present disclosure, and thus should not be considered as limitation to the scope, and those ordinarily skilled in the art still could obtain other relevant drawings according to the drawings, without using any inventive efforts.

FIG. 1 is a map of an expression cassette expressing a herbicide-resistant gene OE89Y;

FIG. 2 is a structural schematic diagram of a pBI121 vector provided by the present disclosure;

FIG. 3 shows test of OEM89Y-transformed positive soybeans provided by the present disclosure;

FIG. 4 shows growth status of transgenic positive soybeans after glyphosate spraying provided by the present disclosure;

FIG. 5 is a structural diagram of an expression cassette of 4KO89; and

FIG. 6 is a map of an expression cassette expressing herbicide-resistant gene CP4-GOX.

### DETAILED DESCRIPTION OF EMBODIMENTS

Reference will now be made in detail to embodiments of the present disclosure, one or more examples of which are described below. Each example is provided by way of illustration, not limitation to the present disclosure. In fact, it would be apparent to those skilled in the art that various modifications and variations could be made in the present disclosure without departing from the scope or spirit of the present disclosure. For example, features illustrated or described as part of one embodiment may be used in another embodiment to yield a still further embodiment.

Unless defined otherwise, all technical and scientific terms used herein have the same meaning as commonly understood by those of ordinary skill in the art. Although any methods and materials similar or equivalent to those described herein can be used in the practice or testing herein, some methods and materials are now described. Unless otherwise specified, techniques employed or contemplated herein are standard methods. The materials, methods and examples are illustrative only and not intended to be limiting.

Unless otherwise indicated, the practice of the present disclosure will employ conventional technologies of plant physiology, plant molecular genetics, cell biology, molecular biology (including recombination technique), microbiology, biochemistry and immunology, which are within the scope of capabilities of those skilled in the art. Such technologies are explained fully in the literature, such as Molecular Cloning: A Laboratory Manual, 2^{nd} Edition (Sambrook et al., 1989); Oligonucleotide Synthesis (M. J. Gait, ed., 1984); Plant Physiology (Cang Jing, et al., 2017); Methods in Enzymology (Academic Press, Inc.); Handbook of Experimental Immunology (D. M. Weir and C. C. Blackwell, ed.); Current Protocols in Molecular Biology (F. M. Ausubel et al., ed., 1987); Plant Molecular Genetics (Monica A. Hughes, et al.), and PCR: The Polymerase Chain Reaction (Mullis et al., ed., 1994), each of which is expressly incorporated herein by reference.

In order to make the objectives, technical solutions and advantages of the embodiments in the present disclosure clearer, technical solutions in the embodiments of the present disclosure will be described clearly and completely below. Examples, for which no concrete conditions are specified, are carried out according to conventional conditions or conditions recommended by manufactures. If manufacturers of reagents or instruments used are not specified, all of them are conventional products commercially available.

Characteristics and performances of the present disclosure are further described in detail below in conjunction with examples.

### Example 1

The present example provides an EPSPS mutant, which was derived from rice and was a rice EPSPS mutant OE89Y, and which was obtained after mutation of a wild-type rice EPSPS (with an amino acid sequence as set forth in SEQ ID NO: 2), where an amino acid sequence thereof is as set forth in SEQ ID NO: 4.

Compared with the amino acid sequence of the wild-type rice EPSPS as set forth in SEQ ID NO: 2, the rice EPSPS mutant OE89Y provided by the present example had A131G, G172A, P177S, K274R, K313E and V403A mutations.

That is, compared with the wild-type rice EPSPS, an amino acid residue at site 131 of the rice EPSPS mutant OE89Y was mutated from A to G, which site is corresponding to site 59 of E. coli EPSPS; an amino acid residue at site 172 was mutated from G to A, which site is corresponding to site 96 of E. coli EPSPS; an amino acid residue at site 177 was mutated from P to S, which site is corresponding to site 101 of E. coli EPSPS; an amino acid residue at site 274 was mutated from K to R, which site is corresponding to site 194 of E. coli EPSPS; an amino acid residue at site 313 was mutated from K to E, which site is corresponding to site 232 of E. coli EPSPS; and an amino acid residue at site 403 was mutated from V to A, which site is corresponding to site 314 of E. coli EPSPS.

The present example further provides a coding gene encoding the above rice EPSPS mutant OE89Y, a nucleotide sequence of which is as set forth in SEQ ID NO: 3. In order to improve translational efficiency of the coding gene of OE89Y in soybean, the nucleotide sequence was optimized based on the codon bias of soybean nucleotide sequence, where the optimized nucleotide sequence (SEQ ID NO: 3) of OE89Y had 77% homology to an original wild-type rice EPSPS.

The coding gene of the rice EPSPS mutant OE89Y provided by the present example of the present disclosure can be obtained through a chemical synthesis method.

### Example 2

### Construction of Optimized OE89Y Expression Cassette and Expression Vector.

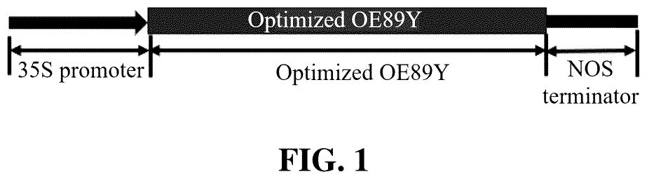

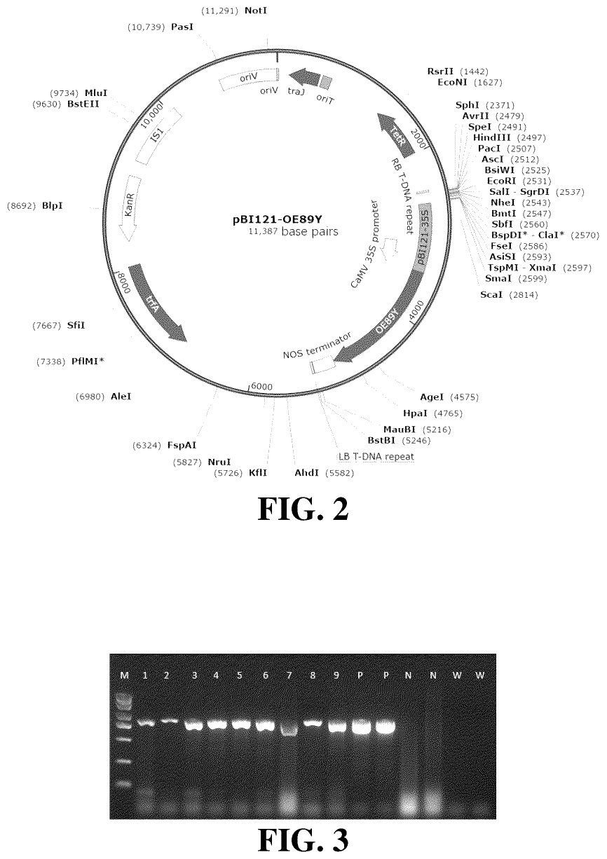

An optimized OE89Y gene was synthesized by a chemical synthesis method, and then primers were designed to amplify an OE89Y gene fragment with an overlapping fragment of 35S and NOS. 35S promoter was obtained through amplification from a pBI121 vector. 5′-end of the OE89Y gene fragment was linked to 35S by a general method in molecular biology, and then linked to a NOS terminator at 3′-end, to form an expression cassette which enables expression in plants (5′-end thereof had a BmHI site, and 3′-end had a BstBI site). A structure of the expression cassette is as shown in FIG. 1. The expression cassette was linked to a modified pBI121 vector by a homologous recombination method, to obtain a T-DNA vector pBI121-OE89Y, with a vector structure thereof as shown in FIG. 2.

### Example 3

The present example provides use of the above OE89Y expression cassette in cultivating glyphosate-resistant soybean, including following steps.

### 1. Agrobacterium Preparation

pBI121-OE89Y vector containing strains 400 μl stored at −80° C. were coated onto YEP

(LB) medium, and cultured for about 24 hours.

### 2. Seed Disinfection and Soak

Soybean seeds were sterilized by a chlorine gas dry method, and sterilized soybean seeds were soaked with water for 20-24 hours.

### 3. Infection and Co-Culturing

To an infection solution, GA3 (gibberellin), 6-BA (6-benzylaminopurine), L-Cys (L-cysteine), and AS (acetosyringone) were added for later use.

Imbibed soybean seeds were transferred from water to a sterile Petri dish. Seed coat was removed, radicle was cut off, and the seeds were cut longitudinally along hilum with scalpel, to separate cotyledon and hypocotyl uniformly into two parts, as explants for later use.

Agrobacterium cultured for 24 hours was added to the prepared infection solution, and OD₆₀₀ of bacterial solution was adjusted to 0.7-0.8.

The soybean explants were poured into a sterilized cup, and washed once with clean water, and the water was blotted up. The prepared Agrobacterium was added into the cup with the soybean explants, followed by infection at room temperature for no less than 1 hour. To the sterile Petri dish containing sterile filter paper, 6 ml of co-culture medium (infection solution+DTT) was added, and the infected soybean explants were placed in the above Petri dish with growing point facing down. After sealing, culture was performed at 24° C. in light for 4-5 days, with 16 h in light/8 h in dark.

### 4. Screening

Elongated hypocotyls of the co-cultured cotyledons were dissected in half, and obvious budlets were removed. The explants were transferred to a bud inducing medium (SIM g12), 10 explants/dish, and cultured at 24° C. for 3 weeks, with 18 h in light/6 h in dark.

### 5. Bud Elongation I

Etiolated cotyledons of the explants were removed, and the explants with shoot bud were transferred to the bud elongation medium (SEM g3 Petri dish), 6 explants/dish, and cultured at 24° C. for 2 weeks, with a cycle of 18 h in light/6 h in dark.

### 6. Bud Elongation II

Blackened basal parts of the explants with significant elongation were excised to expose tissues that can absorb nutrients, cotyledons and majority of budlets were removed, followed by transfer to the bud elongation medium (SEM g2 cup), 4-6 explants/cup, and cultured for 2 weeks, followed by subculture in a fresh medium.

### 7. Rooting

Seedlings elongated to exceed ⅔ of the culture cup were cut off, transferred to a rooting medium, and cultured at 24° C. with a cycle of 18 h in light/6 h in dark. After strong roots were induced, the seedlings were taken out, and cleaned to remove root medium. Plants were numbered, transplanted into nursery pots, further cultured for about 10 d, and then transferred into a greenhouse.

### Example 4

In the present example, positive plants were obtained and identified.

After one week of transplantation of CO generation seedlings provided by Example 3, glyphosate at 10× field dosage was sprayed. The present test sprayed Roundup herbicide (41% glyphosate isopropylammonium salt aqueous solution). After one week of spraying, survival rice seedlings were selected and sampled. DNA was extracted by an SLS method. Designed specific primers were used for transgene detection. Primer sequences are as follows:

Gm2M89-F:
(SEQ ID NO: 27)
GGAAATGCTTATGTTGAAGGAGATGCA;
and
Gm2M89-R:
(SEQ ID NO: 28)
TTCGAAGATCTAGTAACATAGATGACACCG.

PCR was performed using MIX from Nanjing Vazyme Biotech Co., Ltd., and a PCR reaction system (20 μl) is as follows:

| 2x MIX buffer | 10 | μl; |
|---|---|---|
| 10 μM Gm2M89-F | 1 | μl; |
| 10 μM Gm2M89-R | 1 | μl; |
| genomic DNA | 1 μl; and | |
| water | 7 | μl. |

PCR reaction conditions are as follows:

| | | | | | |
|---|---|---|---|---|---|
| 94° | C. | 3 | min | | |
| 94° | C. | 15 | s | | |
| 58° | C. | 15 | s | {close oversize brace} | 30 cycles |
| 72° | C. | 20 | s | | |
| 72° | C. | 5 | min | | |
| 4° | C. | 2 | min | | |

3 μl of PCR product was taken, and ran on 1% agarose gel for electrophoresis, where a size of a target band was 862 bp. FIG. 3 shows that 9 different OE89Y gene transformed plants all had integration of target gene.

Lane M is DNA molecular weight marker DL2000, Lane N is wild-type soybean negative control, P is plasmid DNA positive control, W is water, Lanes 1-9 are different transformation events, and amplification of specific fragments with a size about 862 bp indicated a transgenic positive plant containing the target gene.

### Example 5

### Testing of Herbicide Tolerance of Positive Plants.

The present example took wild-type soybean plants as controls, and positive soybean plants transformed with the OE89Y gene obtained in Example 4 as experimental groups, to verify glyphosate resistance of the OE89Y gene in soybeans.

An experimental method is as follows.

The positive soybean plants transformed with the OE89Y gene, 10 cm-15 cm tall and consistent in growth status, and wild-type soybean plants with a similar size were uniformly arranged in the same experimental area (avoiding leaf overlapping), and then sprayed with glyphosate at 10 times field dosage (commercially available 41% glyphosate isopropylammonium salt solvent). After leaf surfaces were dry, the plants in the experimental group and the control group were moved into a greenhouse to culture, and photographed one week later, to record growth status of the plants.

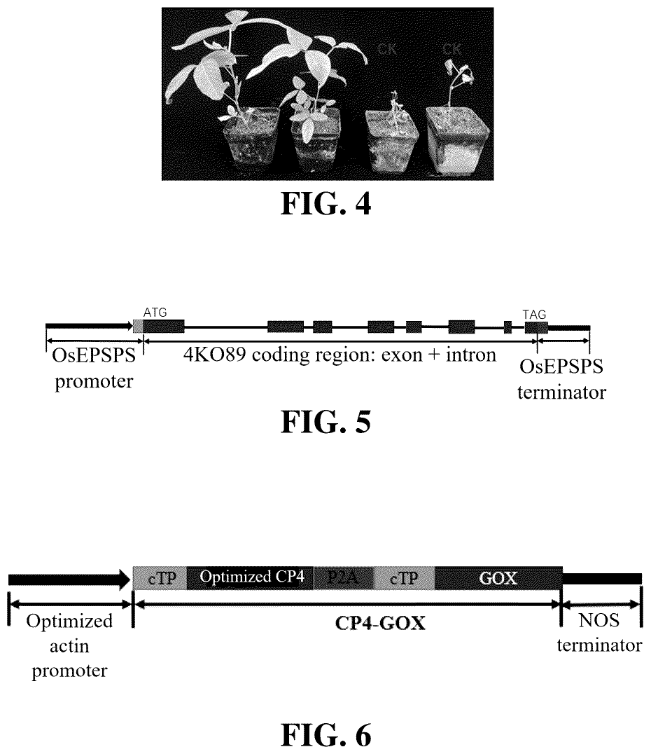

It can be seen from FIG. 4 that after glyphosate at 10 times the field dosage was sprayed, the wild-type soybeans were not resistant to glyphosate and died, while growth of the soybeans transformed with the OE89Y gene under the same treatment was hardly affected, and transgenic soybeans with sufficient glyphosate resistance can be obtained. The above outcome demonstrates that the optimized OE89Y gene can significantly improve the glyphosate resistance of the transgenic soybeans.

The above-mentioned are merely for preferred embodiments of the present disclosure, and it should be indicated that those ordinarily skilled in the art further could make improvements and modifications, without departing from the principle of the present disclosure, and all of these improvements and modifications also should be considered as the scope of protection of the present disclosure.

### Example 6

The present example provides an expression cassette (designated 4KO89) enabling expression of mutation sites of an EPSPS enzyme, including A131G, G172A, P177S, K274R, K313E and V403A.

The present example constructed the 4KO89 expression cassette, where a base sequence thereof is as set forth in SEQ ID NO: 8, and an amino acid sequence of the EPSPS enzyme mutant is as set forth in SEQ ID NO: 4, with a structure as shown in FIG. 5. The expression cassette consisted of an OsEPSPS gene promoter, a 4KO89 coding gene (SEQ ID NO: 5) and an OsEPSPS terminator.

Compared with a nucleotide sequence (SEQ ID NO: 9) of the coding gene of the wild-type rice EPSPS (4KOWT), it had multiple base mutations in exons, that is, from 5′-end to 3′-end of the nucleic acid sequence of the mutant 4KO89, a base at site 3096 was mutated from C to G, two consecutive bases at sites 3219 and 3220 were mutated from GA to CG, site 3233 was mutated from C to T, site 3235 was mutated from A to C, site 3886 was mutated from A to G, site 4002 was mutated from A to G, and site 4582 was mutated from T to C. Compared with the amino acid sequence (SEQ ID NO: 2) of the wild-type rice EPSPS, the amino acid sequence thereof had site mutations of multiple amino acid residues, that is, from terminal N to terminal C, the amino acid residue at site 131 was mutated from A to G, the amino acid residue at site 172 was mutated from G to A, the amino acid residue at site 177 was mutated from P to S, the amino acid residue at site 274 was mutated from K to R, the amino acid residue at site 313 was mutated from K to E, and the amino acid residue at site 403 was mutated from V to A, respectively.

The multiple site mutations rendered the rice EPSPS full-length gene mutant 4KO89 to be resistant to glyphosate.

### Example 7

The present example transformed rice by a particle bombardment method, specifically including following steps.

### 1. Callus Induction

1.1 Seed Disinfection

Dry mature seeds were taken and manually unshelled. Plump seeds without bacterial plaque were selected, and sterilized for 20-60 seconds by adding 60%-80% alcohol. The alcohol was poured out, and the seeds were cleaned once with sterile water. 2-3% sodium hypochlorite solution was added for soak and sterilization for 15-20 minutes. The sodium hypochlorite solution was poured out, and the seeds were soaked and cleaned with sterile water for 6-7 times, 3 minutes each time.
1.2 Induction and Subculture

The seeds were blotted up on sterile filter paper, and put in an induction medium, with 10-15 seeds in each dish. After operation was completed, Petri dishes were sealed with a parafilm, followed by culture at 30° C. in dark for 28-35 days. Calli were transferred into a fresh medium, and further cultured for about 7-14 days.
1.3 Hypertonic Treatment

Globular calli with appropriate size were transferred to a hypertonic medium and cultured. The calli in each dish were placed within a central circular area of 2.5-3 cm in diameter and cultured for 4-8 hours.

### 2. DNA Fragment Wrapping

2.1 Preparation of DNA Fragment

OsEPSPS gene fragment solution and CP4-EPSPS gene fragment solution provided by Example 6 were adjusted to a concentration of 50-1,000 ng/μL for later use.
2.2 Preparation of Gold Powder Mother Liquor (prepared immediately before use)

30 mg of microparticles were weighed out and put into a 1.5 ml microfuge tube. 1 ml of 70% ethanol (v/v) was added. Mixture was vigorously vortexed and shaken for 3-5 minutes. The microparticles were soaked in 70% ethanol for 15 minutes. The microparticles were made into pellets by rotating in a microfuge for 5 seconds. Supernatant was discarded.

Following washing steps were repeated three times: adding 1 ml of sterile water; vigorously vortexing for 1 minute; and letting the microparticles to settle for 1 minute. The microparticles were made into pellets by rotating briefly in the microfuge, and liquid was aspirated and discarded. After a third wash, 500 μl of sterile 50% glycerol was added to bring a microparticle concentration to 60 mg/ml (assuming no loss during preparation). 60 mg (0.8-1.5 μm in diameter) of gold powder was weighed into a 1.5 ml Eppendorf tube; 0.5 ml of 100% ethanol was added, and mixture was thoroughly vortexed for 3-5 min, or ultrasonically well mixed, allowed to stand for 5 min, and centrifuged at 5,000 rpm for 30 s; the steps were repeated 2-3 times; 0.5 mL of 75% ethanol was added, and mixture was thoroughly well vortexed, allowed to stand for 1 min, and centrifuged at 5,000 rpm for 30 s; supernatant was discarded, 1.2 ml of sterile 50% glycerol was added, and mixture was thoroughly vortexed, split charging into 50 μL/1.5 ml Eppendorf tubes, and stored for up to 2 weeks at 4° C.
2.3 DNA Encapsulation

50 μl of gold powder suspension (referring to the gold powder mother liquor in 1.5 ml tube) was taken and sonicated using a sonifier for 1-2 min, and then vortexed thoroughly. Then 5 μl of DNA (50-1,000 ng/μl), 50 μl of 2.5 M CaCl₂) and 20 μl of 0.1 M spermidine were added in sequence, while vortexing, where once a substance was added, shaking can be performed for about 30 s. Mixed sample was thoroughly vortexed, allowed to stand on ice for 3-10 min, and centrifuged at 3,000-10,000 rpm for 10-30 s, and supernatant was removed. 140 μl of 70% ethanol was added, mixture was vortexed and mixed well, allowed to stand for 1-2 min, and centrifuged at 3,000-10,000 rpm for 10-30 s, and supernatant was removed. 140 μl of 70% ethanol was added again, mixture was vortexed and mixed well, allowed to stand for 1-2 min, and centrifuged at 3,000-10,000 rpm for 10-30 s, and supernatant was removed. 140 μl of 100% ethanol was added, mixture was vortexed and mixed well, allowed to stand for 1-2 min, and centrifuged at 3,000-10,000 rpm for 10-30 s, and supernatant was removed. 140 μl of 100% ethanol was added again, mixture was vortexed and mixed well, allowed to stand for 1-2 min, and centrifuged at 5,000 rpm for 10-30 s, and supernatant was removed. Finally, resultant was vortexed and mixed well with 30-100 μl of anhydrous ethanol, placed on ice until use, and left for no more than 4 h.

The prepared gold powder+OsEPSPS gene suspension or CP4-GOX gene suspension was uniformly spotted onto a carrier film, and allowed to air-dry at room temperature before bombarding rice calli.

### 3. Microparticle Bombardment

3.1 Operation Preparatory to Bombardment

Before use, the carrier film, a carrier film fixing groove, a breakage film, a stainless steel net, a breakage film lid, a microparticle emission device and the like were soaked in 75% alcohol for 15 minutes and then dried in air for later use.
3.2 Particle Bombardment Transformation

Rice calli having been treated for 4-8 h on the hypertonic medium were bombarded with the biolistics.

Parameters for bombardment with the biolistics were: the breakage film of 6-7 MPa, a vacuum degree of −0.09 MPa, and a bombarding distance of 5-7 cm.

Following bombardment, the calli were further cultured on the hypertonic medium for 12-24 h.

### 4. Culture and Rooting

4.1 The calli were transferred to a screening medium S1g400, and cultured at 30° C. in dark for 14 days.
4.2 The calli were transferred to a screening medium S2g400, and cultured at 30° C. in dark for 14 days.
4.3 The calli were transferred to a differentiation medium Fg5, and cultured at 30° C. in light of 18 hours, for about 21 days, and then changed into a fresh differentiation medium, and further cultured for about 21 days.
4.4 Primordia and young buds were picked out to a fresh differentiation medium Fg5, and further cultured at 30° C. for about 21 days.
4.5 When growing to about 2 cm, newborn seedlings were transferred to a rooting culture medium to be cultured at 30° C. in light of 18 hours for 2-4 weeks.
4.6 After roots were induced and the seedlings grew to 7-10 cm, the seedlings were transplanted to soil, which seedlings were CO generation.

### Example 8

In the present example, positive plants were obtained and identified.

After one week of transplantation of the CO generation seedlings provided by Example 7, glyphosate at 10× field dosage was sprayed. The present test sprayed Roundup herbicide (41% glyphosate isopropylammonium salt aqueous solution). After one week of spraying, survival rice seedlings were selected and sampled. DNA was extracted by an alkali cooking method. Designed specific primers were used for detecting copy number of EPSPS. Primer sequences are as follows:

R96A5:
(SEQ ID NO: 25)
CAAGCAGTGCTTTCTCCCAAAATTATG;
and
R96A3:
(SEQ ID NO: 26)
AATGCTAATTCAAAAGAAGACATCAAGACC.

PCR used KOD enzyme from TOYOBO CO., LTD., and PCR reaction system (20 μl) is as follows:

| | | |
|---|---|---|
| 2X KOD buffer | 10 | μl |
| 10 μM R96A5 | 1 | μl |
| 10 μM R96A3 | 1 | μl |
| genomic DNA | 1 | μl |
| KOD enzyme | 0.2 | μl |
| water | 6.8 | μl. |

PCR reaction conditions are as follows:

| | | | | | |
|---|---|---|---|---|---|
| 94° | C. | 3 | min; | | |
| 98° | C. | 15 | s; | | |
| 60° | C. | 8 | s; | {close oversize brace} | 30 cycles |
| 68° | C. | 30 | s; | | |
| 68° | C. | 5 | min; | | |
| 4° | C. | 2 | min. | | |

3 μl of PCR product was taken, and ran on 1% agarose gel for electrophoresis, where a size of a target band was 1063 bp. The PCR product was sent to Sangon Biotech (Shanghai) Co., Ltd. for sequencing. The copy number of the CO generation seedlings was evaluated based on a sequencing result, and the CO generation seedlings with a low copy number were retained.

### Comparative Example 1

The present comparative example provides an expression cassette (designated CP4-GOX) capable of expressing CP4 gene and GOX gene.

Primers were designed using a commercially available pBI121 vector as a template, to amplify a NOS terminator fragment. Sangon Biotech (Shanghai) Co., Ltd. artificially synthesized optimized rice actin promoter, optimized cTP (chloroplast signal peptide), optimized CP4-EPSPS gene fragment, and GOX gene (glyphosate oxidoreductase gene) fragment. The primers were designed, and various fragments were spliced into a CP4-GOX coding gene fragment by overlapping PCR, to obtain the CP4-GOX expression cassette, with a structure as shown in FIG. 6. The expression cassette consisted of actin promoter, CP4-EPSPS gene, GOX gene and NOS terminator.

### Experimental Example 1

### 1. Experiment of Testing Herbicide Tolerance of Positive Plants.

After one week of spraying of glyphosate at 10 times the field dosage (a normal field dosage (1 time) of glyphosate is 1060 grams of active ingredients per hectare, and 10 times is 10,600 grams of active ingredients per hectare) for the CO generation seedlings of Example 7, the number of surviving plants and the number of dead plants were counted, with statistical data as listed in Table 1.

TABLE 1
Number of C0 Generation Plants at least
Resistant to 10 times Glyphosate

| DNA | Number of Plants |
|---|---|
| Wild type | 0 |
| Example 6 | >300 |

Table 1 shows that upon transformation of the 4KO89 expression cassette in Example 6 into rice, the rice CO generation seedlings had the highest survival rate. 168 and 118 CO generation seedlings transformed with the 4KO89 and CP4-GOX expression cassettes (Comparative Example 1) were then sprayed with 10 times glyphosate. Statistical data was recorded 14 days later. 3 CO generation seedlings with a low copy number and good growth determined by the above method were transplanted, remaining plants were further sprayed with 20 times glyphosate (the normal field use dosage (1 time) of glyphosate is 1060 grams of active ingredients per hectare, and 20 times is 21,200 grams of active ingredients per hectare). Statistical data was recorded 14 days later. Surviving seedlings were further sprayed with 50 times glyphosate, and statistical data was recorded 14 days later. The statistical data is listed in Table 2.

TABLE 2
Statistical Results after 14 Days of Spraying of Different Glyphosate Dosages

| | | 10 Times | | 20 Times | | 50 Times | |
|---|---|---|---|---|---|---|---|
| | | Moderate-to- | High | Moderate-to- | High | Moderate-to- | High |
| | Number of | high | tolerance | high | tolerance | high | tolerance |
| DNA | Seedlings | tolerance % | % | tolerance % | % | tolerance % | % |
| CP4-GOX | 118 | 12.7 | 2.5 | 0 | 0 | 0 | 0 |
| Example 6 | 169 | 52.1 | 5.3 | 14.8 | 1.2 | 1.8 | 0 |

At present, CP4-EPSPS is the most widely used glyphosate-resistant gene in agriculture, and the GOX gene can degrade glyphosate. However, it can be seen from Table 2 that after spraying of 10 times glyphosate, proportions of moderate-to-high tolerance (plants slightly affected) and high tolerance (plants almost unaffected) of the rice seedlings transformed with the CP4-GOX were 12.7% and 2.5%, respectively, while corresponding proportions of the rice seedlings transformed with the 4KO89 expression cassette in Example 6 were 52.1% and 5.3%. After spraying of 20 times glyphosate, the rice seedlings transformed with the CP4-GOX were all dead, but 1.8% of the rice seedlings transformed with the 4KO89 still achieved moderate-to-high tolerance under the spraying of 50 times glyphosate.

The above outcome indicates that the 4KO89 expression cassette is capable of conferring resistance to at least 10 times glyphosate concentration on the rice plants, and the resistance of the 4KO89 expression cassette is much higher than superimposed resistance of the existing glyphosate-resistant genes CP4 and GOX, and exhibits excellent glyphosate resistance. Moreover, the rice plants transformed with this expression cassette can grow normally, indicating that the bio-enzyme activity of the rice EPSPS mutant in this expression cassette is normal.

The above-mentioned are merely for preferred embodiments of the present disclosure and not intended to limit the present disclosure. For those skilled in the art, various modifications and changes could be made to the present disclosure. Any amendments, equivalent replacements, improvements and so on, made within the spirit and principle of the present disclosure, should be covered within the scope of protection of the present disclosure.

1. An EPSPS enzyme mutant, resistant to glyphosate, wherein the EPSPS enzyme mutant is:
obtained by mutating amino acids at sites 131, 172, 177, 274, 313 and 403 of a rice-derived wild-type EPSPS enzyme, wherein an amino acid sequence of the wild-type EPSPS enzyme is as set forth in SEQ ID NO: 2; and
amino acids at sites 131, 172, 177, 274, 313 and 403 of the EPSPS enzyme mutant are G, A, S, R, E and A, respectively.

2. A nucleic acid molecule, encoding the EPSPS enzyme mutant according to claim 1.

3. The nucleic acid molecule according to claim 2, wherein a nucleotide sequence of the nucleic acid molecule is as set forth in SEQ ID NO: 3.

4. A nucleic acid molecule, wherein a sequence of the nucleic acid molecule is as set forth in SEQ ID NO: 5.

5. An expression cassette, comprising the nucleic acid molecule according to claim 4.

6. The expression cassette according to claim 5, wherein the expression cassette further comprises a promoter and a terminator.

7. The expression cassette according to claim 6, wherein when the nucleic acid molecule of the expression cassette is the nucleic acid molecule as set forth in SEQ ID NO: 5, a sequence of the promoter of the expression cassette is as set forth in SEQ ID NO: 6.

8. The expression cassette according to claim 6, wherein when the nucleic acid molecule of the expression cassette is the nucleic acid molecule as set forth in SEQ ID NO: 5, a sequence of the terminator of the expression cassette is as set forth in SEQ ID NO: 7.

9. An expression vector, comprising the expression cassette according to claim 5.

10. A recombinant bacterium, wherein the recombinant bacterium contains a coding gene encoding the EPSPS enzyme mutant according to claim 1.

11. The recombinant bacterium according to claim 10, wherein the recombinant bacterium is a bacterium or a fungus.

12. The recombinant bacterium according to claim 11, wherein the bacterium is Agrobacterium or E. coli; and the fungus is yeast.

13. Use of the EPSPS enzyme mutant according to claim 1 in cultivating a glyphosate-resistant plant, wherein the plant is rice or soybean.

14. The use according to claim 13, comprising at least one of following use modes:
(1) delivering the nucleic acid molecule into a cell of a plant of interest;
(2) introducing the expression cassette into a plant of interest by particle bombardment or an Agrobacterium-mediated transformation method;
(3) transforming a plant of interest with the vector, wherein the vector contains a coding gene encoding a glyphosate-resistant rice EPSPS enzyme mutant;
(4) introducing the recombinant bacterium into a plant of interest, wherein the recombinant bacterium contains a coding gene encoding a glyphosate-resistant rice EPSPS enzyme mutant; and
(5) performing gene editing on a cell of a plant of interest, so as to encode the EPSPS enzyme mutant resistant to glyphosate, wherein the EPSPS enzyme mutant is:
obtained by mutating amino acids at sites 131, 172, 177, 274, 313 and 403 of a rice-derived wild-type EPSPS enzyme, wherein an amino acid sequence of the wild-type EPSPS enzyme is as set forth in SEQ ID NO: 2; and
the amino acids at the sites 131, 172, 177, 274, 313 and 403 of the EPSPS enzyme mutant are G, A, S, R, E and A, respectively.

15. A method for cultivating a glyphosate-resistant plant, wherein an EPSPS enzyme in a cell of a plant of interest and a rice-derived wild-type EPSPS enzyme are subjected to sequence alignment, so as to enable mutation of amino acids of the EPSPS enzyme of the cell of the plant of interest corresponding to sites 131, 172, 177, 274, 313 and 403 of the rice-derived wild-type EPSPS enzyme into G, A, S, R, E and A, respectively, wherein an amino acid sequence of the wild-type EPSPS enzyme is as set forth in SEQ ID NO: 2; and the plant of interest is rice or soybean.

16. A method for detecting whether a plant to be tested is an EPSPS mutant plant, comprising: determining whether the plant to be tested contains the EPSPS enzyme mutant according to claim 1.


SEQUENCE LISTING (28 sequences)

SEQ ID NO: 1 - DNA, 1548 bp, synthetic construct
  source 1..1548: mol_type = other DNA
atggcggcgaccatggcgtccaacgccgcggctgcggcggcggtgtccctggaccaggccgtggcggcgtcggcggcgttctcgtcgcggaagcagctgcggctgcccgccgcggcgcgcggggggatgcgggtgcgggtgcgggcgcgggggcggcgggaggcggtggtggtggcgtccgcgtcgtcgtcgtcggtggcagcgccggcggcgaaggcggaggagatcgtgctccagcccatcagggagatctccggggcggttcagctgccagggtccaagtcgctctccaacaggatcctcctcctctccgccctctccgagggcacaacagtggtggacaacttgctgaacagtgaggatgttcactacatgcttgaggccctgaaagccctcgggctctctgtggaagcagataaagttgcaaaaagagctgtagtcgttggctgtggtggcaagtttcctgttgagaaggatgcgaaagaggaagtgcaactcttcttggggaacgctggaactgcaatgcgaccattgacagcagccgtgactgctgctggtggaaatgcaacttatgtgcttgatggagtgccacgaatgagggagagaccgattggtgacttggttgtcgggttgaaacaacttggtgcggatgtcgactgtttccttggcactgaatgcccacctgttcgtgtcaagggaattggaggacttcctggtggcaaggttaagctctctggttccatcagcagtcagtacttgagtgccttgctgatggctgctcctttggcccttggggatgtggagatcgaaatcattgacaaactaatctccattccttacgttgaaatgacattgagattgatggagcgttttggtgtgaaggcagagcattctgatagttgggacagattctatattaagggagggcagaagtacaaatctcctggaaatgcctatgttgaaggtgatgcctcaagcgcgagctatttcttggctggtgctgcaatcactggaggcactgtgacagttcaaggttgtggtacgaccagtttgcagggtgatgtcaaatttgctgaggtacttgagatgatgggagcaaaggttacatggactgacaccagtgtaaccgtaactggtccaccacgtgagccttatgggaagaaacacctgaaagctgttgatgtcaacatgaacaaaatgcctgatgttgccatgacccttgccgttgttgcactcttcgctgatggtccaactgctatcagagatgtggcttcctggagagtaaaggaaaccgaaaggatggttgcaattcggaccgagctaacaaagctgggagcatcggttgaagaaggtcctgactactgcatcatcaccccaccggagaagctgaacatcacggcaatcgacacctacgatgatcacaggatggccatggccttctccctcgctgcctgcgccgacgtgcccgtgacgatcagggaccctggttgcacccgcaagaccttccccaactacttcgacgttctaagcactttcgtcaggaactga

SEQ ID NO: 2 - protein, 515 aa, synthetic construct
MAATMASNAAAAAAVSLDQAVAASAAFSSRKQLRLPAAARGGMRVRVRARGRREAVVVASASSSSVAAPAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAGTAMRPLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDVAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 3 - DNA, 1548 bp, synthetic construct
  source 1..1548: mol_type = other DNA
atggcggcgaccatggcatccaacgccgcggctgcagccgccgttagcctagaccaagctgttgctgcaagcgccgcattttctagtaggaagcaacttcggttgccagcagctgctagagggggtatgagggtgcgtgttagagctcgggggcgcagggaggcagttgtagtagcctcagcttcttccagtagtgtggccgcaccggcagcaaaggctgaggaaattgtgctgcagccaataagggagatttcaggggcggtgcagctgcctggcagcaaatctctgtcaaatcgaatactcctacttagcgctctctctgagggaacgaccgttgtggacaatttgttgaactccgaagacgttcactacatgcttgaggctctgaagggcctgggattgagtgtcgaagcagacaaggtggctaaacgggctgtagtcgtcggctgtggtggcaaattccctgtggagaaagatgctaaagaagaagttcaactttttttaggtaacgcagcgacagcaatgagatcgctgactgctgctgttactgcggctggtggaaatgccacctatgtcttggatggtgttccaagaatgcgtgaaaggcccattggtgatcttgttgttggtctcaaacagcttggagcagatgttgactgttttcttgggacagaatgccctcctgtcagggtaaagggcatcggcgggctacccggtgggaaggttaagttatctggctcaatttcttcacaatatctcagtgcattattgatggctgcacctttggctcttggagatgtggaaatcgagataattgatcgtcttatctccattccatacgttgagatgactcttaggttgatggagcgttttggagttaaagcagaacatagtgattcttgggatagattctacatcaaaggaggtcagaagtatgaatctccaggaaatgcttatgttgaaggagatgcatcaagtgcatcctacttccttgctggggctgccatcacagggggaactgtgacggtgcaaggatgtggaaccacttcgttgcaaggtgatgtgaaatttgctgaagttttggaaatgatgggtgcaaaagttacatggactgatacttctgtcactgtcaccggtcctcctcgagagccatatggaaagaagcatttaaaagctgtagacgtgaacatgaataagatgcctgatgcagctatgacactcgcagttgttgctctatttgctgatggaccgacggccataagagatgttgcgtcatggagagtgaaggaaactgaaaggatggtggctataagaacagagttaacaaaactcggtgcctcggttgaggagggccccgattattgcattattaccccacccgaaaagttgaatataacagctattgacacatatgatgatcatcgaatggccatggcattctccctggctgcatgtgctgatgtaccagtaaccattagggatcctggttgcactcgcaaaactttcccaaattactttgacgtcctatcaacatttgtaagaaactga

SEQ ID NO: 4 - protein, 515 aa, synthetic construct
MAATMASNAAAAAAVSLDQAVAASAAFSSRKQLRLPAAARGGMRVRVRARGRREAVVVASASSSSVAAPAAKAEEIVLQPIREISGAVQLPGSKSLSNRILLLSALSEGTTVVDNLLNSEDVHYMLEALKGLGLSVEADKVAKRAVVVGCGGKFPVEKDAKEEVQLFLGNAATAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDRLISIPYVEMTLRLMERFGVKAEHSDSWDRFYIKGGQKYESPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 5 - DNA, 3270 bp, synthetic construct
  source 1..3270: mol_type = other DNA
atggcggcgaccatggcgtccaacgccgcggctgcggcggcggtgtccctggaccaggccgtggcggcgtcggcggcgttctcgtcgcggaagcagctgcggctgcccgccgcggcgcgcggggggatgcgggtgcgggtgcgggcgcgggggcggcgggaggcggtggtggtggcgtccgcgtcgtcgtcgtcggtggcagcgccggcggcgaaggcggaggagatcgtgctccagcccatcagggagatctccggggcggttcagctgccagggtccaagtcgctctccaacaggatcctcctcctctccgccctctccgaggtgagacgcggatcccttcctcttgcgtgaattccatttctggagatgagattttagggggtttattaggtgaggtggctgtgtttgtgaaatcctaggaattatctctcaagtcaatctaacgatgagatataactgaggttctggttttaatcacacactcatataaccaatttattgaaacattttggtttggcataagaaactgcttacgaaggtatgatatcctcctacatgtcaggctactaaattttcacgacggtatgatccactcaaaacaagtttcttaacgagtctggtgaggtctgttatgaaatttgtgtaaactaaggcaactttggaggtttcgcactgtaccaatgttatgtttgaacattttgcaagcagtgctttctcccaaaattatgcaattttgaggctcctctacatcattataattccccaatacattgctctttattcttaatagctttgatcgcgaaatttaacattttaattcttgagctgttattttgtagcatcagtttatcatgagccatgtttggtactaaatatacaatcccttgggtttatttgtttccaagcatgtcattaacttatcttaatgtggacaagaaactgatgcctgcttacattgctattatttcaagcgggtattgatcctttgacatgtgattgatcatttttttttctctggttattagggcacaacagtggtggacaacttgctgaacagtgaggatgttcactacatgcttgaggccctgaaaggcctcgggctctctgtggaagcagataaagttgcaaaaagagctgtagtcgttggctgtggtggcaagtttcctgttgagaaggatgcgaaagaggaagtgcaactcttcttggggaacgctgcgactgcaatgcgatccttgacagcagccgtgactgctgctggtggaaatgcaacgtatgttttttttttttttaatgtttatgaaaatatgtatggaattcatggggtatgttttatgacctttttctttaccatcagttatgtgcttgatggagtgccacgaatgagggagagaccgattggtgacttggttgtcgggttgaaacaacttggtgcggatgtcgactgtttccttggcactgaatgcccacctgttcgtgtcaagggaattggaggacttcctggtggcaaggttagttactcctaaactgcatcctttgtacttctgtatgcacctcaattctttgtcaaccttctgcatttataaggaacattctatgatgcaattcgaccttacactgcacagtaacttgaaatgtttcatgcttaatcaatatgccatattcctgccaagctcaagcgagcaatatttgtttgaatttggtaccatatttttgtatatttgggcattcctttttggtcttgatgtcttcttttgaattagcatttaactgaattacactcaacaggttaagctctctggttccatcagcagtcagtacttgagtgccttgctgatggctgctcctttggcccttggggatgtggagatcgaaatcattgacagactaatctccattccttacgttgaaatgacattgagattgatggagcgttttggtgtgaaggcagagcattctgatagttgggacagattctatattaagggagggcagaagtacgagtaagcttctacctgccttactgagctgaattattcgggtgtttatgattaactccctaaactaaccctttttcttttctttggcattgacagatctcctggaaatgcctatgttgaaggtgatgcctcaagcgcgagctatttcttggctggtgctgcaatcactggaggcactgtgacagttcaaggttgtggtacgaccagtttgcaggtataactgtagtgcctgttttgacattctaccgtttagtcaagtttagtcagtagtcacatattcagaatatagcacaatctgtattatgccactgttaatcaaatacgcttgacctagagagtgctatataccctagcttaatcttcaaactaaacagttctcttgtggcttgctgtgctgttatgttccctgacctacatgttaatattacagggtgatgtcaaatttgctgaggtacttgagatgatgggagcaaaggttacatggactgacaccagtgtaaccgtaactggtccaccacgtgagccttatgggaagaaacacctgaaagctgttgatgtcaacatgaacaaaatgcctgatgctgccatgacccttgccgttgttgcactcttcgctgatggtccaactgctatcagagatggtaaacattaaggcctattatacctgttctatcatactagcaattactgcttagcattgtgacaaaacaaataaccaaactttcttcaaaataacttagaaatataagaaaggttcgttttgtgtggtaaacagtactactgtagtttcagctatgaagtttgctgctggcaattttctgaacggtttcagctaaattgcatgtttgttcatcatacttatccattgtcttccacagtggcttcctggagagtaaaggaaaccgaaaggatggttgcaattcggaccgagctaacaaaggtaaattcattaggtcccgtgtcctttcattcttcaagtagtttgttcataagttgaattctccttcaatgatgtttaaattcatcatcttcttttttggtgttgtgccagctgggagcatcggttgaagaaggtcctgactactgcatcatcaccccaccggagaagctgaacatcacggcaatcgacacctacgatgatcacaggatggccatggccttctccctcgctgcctgcgccgacgtgcccgtgacgatcagggaccctggttgcacccgcaagaccttccccaactacttcgacgttctaagcactttcgtcaggaactga

SEQ ID NO: 6 - DNA, 2000 bp, synthetic construct
  source 1..2000: mol_type = other DNA
cactaagccaacatagatatattaggatgcattgatcctggctgctgccacattcaaaagaccaagagcagcacgccatcttgaatttgactctttggttcatgctgggtagcctttcaaagccccaaaagattgggtcgggtctctctttcggctctcgtcggtggctgcttcccacaaagaccgccatcagacgtgaatgaactgcaagtctgcaactacgactccagttgctatccccttaaattactttactactacctttattctaagggccggttcggataattgccaaaatcaatcgtaccaaattttaataatattaggatgaaataaatatgttgcgccaaaattttctactgttactgaaatttggtaacaaactaaacacaaccgtatatttatcaattttaccaaatagtggtatggtttaaaatgacattaatctaaataggtcctgaaatataaaaatgcaccaaccatcaatgcatgcagttacattccaaagttctcctaggacacatatgccatatattttcgatggatagccttaatgtctgactactctcttcatcgcaacaaatctaatataatacaaatctgaataggagcttatataaatatatagtagtagaatatgtcataatattacagtggtattccatattggatttgttgattatagaacgacgaaagtatgcgttaacttacaaggttgtttacattatgacgaaatgaagagaaggaacgcacctactgcgaagagctcaacaactacttcctccaaatttccactcaccaactgatcaccaccaattcacttgtgcagtgccacctacccaatttcattccagtggcgtggtacgtaggttccgcatgtcagtgtgacaatcgtacggactgccatgtgtattgtgttcaggtaggtgcactggtcgtggtctcaccatctctcctccagtcgtacggcgccacgtaggcagtacacgtagacggccttcttcctcctccctcgagttgagttggttggtgagagtgagacaccgacggaacggaaggagaaccacgccgcttggatttttcttttttaccttttcaaattttaatttaaaaaataaaaccattttaaaaacttatcttcaaatacaaatcttttaaaaacactaacacgtgacacacagcgggcacgtcacccaaacgggcgtgacaatattgttttgccacaccaatccagctggtgtggacaaaatgttcatatattgaaaataaaatttaaaacaatttatattttttatctatatcattataaaaattgaagatgtttttaccggtattttgttactcatttgtgcatgagtcggtttttaagtttgttcgcttttggaaatacatatccgtatttgagtatgtttttaagttcgttcgttttttgaaatacaaaaggaatcgtaaaataaatctattttaaaaaactcgcatgctaacttgagacgatcgaactgctaattgcagctcataattttccaaaaaaaaatatatccaaacgagttcttatagtagatttcaccttaattaaaacatataaatgttcacccggtacaacgcacgagtatttttataagtaaaattaaaagtttaaaataaataaaaatcccgccaccacggcgcgatggtaaaagggggacgcttctaaacgggccgggcacgggacgatcggccccgaacccggcccatctaaccgctgtaggcccaccgcccaccaatccaactccgtactacgtgaagcgctggatccgcaacccgttaagcagtccacacgactcgactcgactcgcgcactcgccgtggtaggtggcaacccttcttcctcctctatttcttcttcttcctcccttctccgcctcaccacaccaaccgcaccaaccccaaccccgcgcgcgctctcccctctcccctcccaccaaccccaccccatcctcccgacctccacgccgccggca

SEQ ID NO: 7 - DNA, 1000 bp, synthetic construct
  source 1..1000: mol_type = other DNA
actgagcttttaaaagagtgaggtctaggttctgttgtctgtctgtccatcatccatgtgttgactgttgagggaactcgtttcttcttttcttcacgagatgagtttttgtgtgcctgtaatactagtttgtagcaaaggctgcgttacataaggtgatgagaattgaggtaaaatgagatctgtacactaaattcattcagactgttttggcataaagaataatttggccttctgcgatttcagaagctataaattgccatctcactaaattctccttggtcctcatggcaatgcaacgacagtgtgaagcactgaagcccgtaatgctctatcaccaccatgtacaacagaaccatatatgtccatatgtacaactcgagtgttgtttgagtggccagcaaactggctgaccaagccacacgagagagaatactataaactcaatcatacataacaagcccaagcaacagaagacagaacacaacaacactcgagtctgggaacagcaacgccgatacactacagcatactgcaacgacacaaaggaattggtaaggaaatggcaccaaaatcttgtagatctttagcgagtcattcatttatagcatatgttggaacagacatgagtcacaagatttatgatagcttagataacagatggtcggaattaaccgccgagagcctatagatgaacaaaaactctgttccaccagtgttctcatgctaaaaactttctgaaataccctaccctgttcaccgaatttctccacctgttggatagcagcattttactggtcgctgatggtcacttcaacctccacacctggctcaatggtgattgaggtgatctgcttgacaacatctgcagagctgacaagatcaatcacccttttgtgcacccgcatctcaaaccgatcccaggtgttggtacctgaaacaatgtttacatcattaacatttccaaccgcccatgggagatgcagaacaaagagggcaa

SEQ ID NO: 8 - DNA, 6270 bp, synthetic construct
  source 1..6270: mol_type = other DNA
cactaagccaacatagatatattaggatgcattgatcctggctgctgccacattcaaaagaccaagagcagcacgccatcttgaatttgactctttggttcatgctgggtagcctttcaaagccccaaaagattgggtcgggtctctctttcggctctcgtcggtggctgcttcccacaaagaccgccatcagacgtgaatgaactgcaagtctgcaactacgactccagttgctatccccttaaattactttactactacctttattctaagggccggttcggataattgccaaaatcaatcgtaccaaattttaataatattaggatgaaataaatatgttgcgccaaaattttctactgttactgaaatttggtaacaaactaaacacaaccgtatatttatcaattttaccaaatagtggtatggtttaaaatgacattaatctaaataggtcctgaaatataaaaatgcaccaaccatcaatgcatgcagttacattccaaagttctcctaggacacatatgccatatattttcgatggatagccttaatgtctgactactctcttcatcgcaacaaatctaatataatacaaatctgaataggagcttatataaatatatagtagtagaatatgtcataatattacagtggtattccatattggatttgttgattatagaacgacgaaagtatgcgttaacttacaaggttgtttacattatgacgaaatgaagagaaggaacgcacctactgcgaagagctcaacaactacttcctccaaatttccactcaccaactgatcaccaccaattcacttgtgcagtgccacctacccaatttcattccagtggcgtggtacgtaggttccgcatgtcagtgtgacaatcgtacggactgccatgtgtattgtgttcaggtaggtgcactggtcgtggtctcaccatctctcctccagtcgtacggcgccacgtaggcagtacacgtagacggccttcttcctcctccctcgagttgagttggttggtgagagtgagacaccgacggaacggaaggagaaccacgccgcttggatttttcttttttaccttttcaaattttaatttaaaaaataaaaccattttaaaaacttatcttcaaatacaaatcttttaaaaacactaacacgtgacacacagcgggcacgtcacccaaacgggcgtgacaatattgttttgccacaccaatccagctggtgtggacaaaatgttcatatattgaaaataaaatttaaaacaatttatattttttatctatatcattataaaaattgaagatgtttttaccggtattttgttactcatttgtgcatgagtcggtttttaagtttgttcgcttttggaaatacatatccgtatttgagtatgtttttaagttcgttcgttttttgaaatacaaaaggaatcgtaaaataaatctattttaaaaaactcgcatgctaacttgagacgatcgaactgctaattgcagctcataattttccaaaaaaaaatatatccaaacgagttcttatagtagatttcaccttaattaaaacatataaatgttcacccggtacaacgcacgagtatttttataagtaaaattaaaagtttaaaataaataaaaatcccgccaccacggcgcgatggtaaaagggggacgcttctaaacgggccgggcacgggacgatcggccccgaacccggcccatctaaccgctgtaggcccaccgcccaccaatccaactccgtactacgtgaagcgctggatccgcaacccgttaagcagtccacacgactcgactcgactcgcgcactcgccgtggtaggtggcaacccttcttcctcctctatttcttcttcttcctcccttctccgcctcaccacaccaaccgcaccaaccccaaccccgcgcgcgctctcccctctcccctcccaccaaccccaccccatcctcccgacctccacgccgccggcaatggcggcgaccatggcgtccaacgccgcggctgcggcggcggtgtccctggaccaggccgtggcggcgtcggcggcgttctcgtcgcggaagcagctgcggctgcccgccgcggcgcgcggggggatgcgggtgcgggtgcgggcgcgggggcggcgggaggcggtggtggtggcgtccgcgtcgtcgtcgtcggtggcagcgccggcggcgaaggcggaggagatcgtgctccagcccatcagggagatctccggggcggttcagctgccagggtccaagtcgctctccaacaggatcctcctcctctccgccctctccgaggtgagacgcggatcccttcctcttgcgtgaattccatttctggagatgagattttagggggtttattaggtgaggtggctgtgtttgtgaaatcctaggaattatctctcaagtcaatctaacgatgagatataactgaggttctggttttaatcacacactcatataaccaatttattgaaacattttggtttggcataagaaactgcttacgaaggtatgatatcctcctacatgtcaggctactaaattttcacgacggtatgatccactcaaaacaagtttcttaacgagtctggtgaggtctgttatgaaatttgtgtaaactaaggcaactttggaggtttcgcactgtaccaatgttatgtttgaacattttgcaagcagtgctttctcccaaaattatgcaattttgaggctcctctacatcattataattccccaatacattgctctttattcttaatagctttgatcgcgaaatttaacattttaattcttgagctgttattttgtagcatcagtttatcatgagccatgtttggtactaaatatacaatcccttgggtttatttgtttccaagcatgtcattaacttatcttaatgtggacaagaaactgatgcctgcttacattgctattatttcaagcgggtattgatcctttgacatgtgattgatcatttttttttctctggttattagggcacaacagtggtggacaacttgctgaacagtgaggatgttcactacatgcttgaggccctgaaaggcctcgggctctctgtggaagcagataaagttgcaaaaagagctgtagtcgttggctgtggtggcaagtttcctgttgagaaggatgcgaaagaggaagtgcaactcttcttggggaacgctgcgactgcaatgcgatccttgacagcagccgtgactgctgctggtggaaatgcaacgtatgttttttttttttttaatgtttatgaaaatatgtatggaattcatggggtatgttttatgacctttttctttaccatcagttatgtgcttgatggagtgccacgaatgagggagagaccgattggtgacttggttgtcgggttgaaacaacttggtgcggatgtcgactgtttccttggcactgaatgcccacctgttcgtgtcaagggaattggaggacttcctggtggcaaggttagttactcctaaactgcatcctttgtacttctgtatgcacctcaattctttgtcaaccttctgcatttataaggaacattctatgatgcaattcgaccttacactgcacagtaacttgaaatgtttcatgcttaatcaatatgccatattcctgccaagctcaagcgagcaatatttgtttgaatttggtaccatatttttgtatatttgggcattcctttttggtcttgatgtcttcttttgaattagcatttaactgaattacactcaacaggttaagctctctggttccatcagcagtcagtacttgagtgccttgctgatggctgctcctttggcccttggggatgtggagatcgaaatcattgacagactaatctccattccttacgttgaaatgacattgagattgatggagcgttttggtgtgaaggcagagcattctgatagttgggacagattctatattaagggagggcagaagtacgagtaagcttctacctgccttactgagctgaattattcgggtgtttatgattaactccctaaactaaccctttttcttttctttggcattgacagatctcctggaaatgcctatgttgaaggtgatgcctcaagcgcgagctatttcttggctggtgctgcaatcactggaggcactgtgacagttcaaggttgtggtacgaccagtttgcaggtataactgtagtgcctgttttgacattctaccgtttagtcaagtttagtcagtagtcacatattcagaatatagcacaatctgtattatgccactgttaatcaaatacgcttgacctagagagtgctatataccctagcttaatcttcaaactaaacagttctcttgtggcttgctgtgctgttatgttccctgacctacatgttaatattacagggtgatgtcaaatttgctgaggtacttgagatgatgggagcaaaggttacatggactgacaccagtgtaaccgtaactggtccaccacgtgagccttatgggaagaaacacctgaaagctgttgatgtcaacatgaacaaaatgcctgatgctgccatgacccttgccgttgttgcactcttcgctgatggtccaactgctatcagagatggtaaacattaaggcctattatacctgttctatcatactagcaattactgcttagcattgtgacaaaacaaataaccaaactttcttcaaaataacttagaaatataagaaaggttcgttttgtgtggtaaacagtactactgtagtttcagctatgaagtttgctgctggcaattttctgaacggtttcagctaaattgcatgtttgttcatcatacttatccattgtcttccacagtggcttcctggagagtaaaggaaaccgaaaggatggttgcaattcggaccgagctaacaaaggtaaattcattaggtcccgtgtcctttcattcttcaagtagtttgttcataagttgaattctccttcaatgatgtttaaattcatcatcttcttttttggtgttgtgccagctgggagcatcggttgaagaaggtcctgactactgcatcatcaccccaccggagaagctgaacatcacggcaatcgacacctacgatgatcacaggatggccatggccttctccctcgctgcctgcgccgacgtgcccgtgacgatcagggaccctggttgcacccgcaagaccttccccaactacttcgacgttctaagcactttcgtcaggaactgaactgagcttttaaaagagtgaggtctaggttctgttgtctgtctgtccatcatccatgtgttgactgttgagggaactcgtttcttcttttcttcacgagatgagtttttgtgtgcctgtaatactagtttgtagcaaaggctgcgttacataaggtgatgagaattgaggtaaaatgagatctgtacactaaattcattcagactgttttggcataaagaataatttggccttctgcgatttcagaagctataaattgccatctcactaaattctccttggtcctcatggcaatgcaacgacagtgtgaagcactgaagcccgtaatgctctatcaccaccatgtacaacagaaccatatatgtccatatgtacaactcgagtgttgtttgagtggccagcaaactggctgaccaagccacacgagagagaatactataaactcaatcatacataacaagcccaagcaacagaagacagaacacaacaacactcgagtctgggaacagcaacgccgatacactacagcatactgcaacgacacaaaggaattggtaaggaaatggcaccaaaatcttgtagatctttagcgagtcattcatttatagcatatgttggaacagacatgagtcacaagatttatgatagcttagataacagatggtcggaattaaccgccgagagcctatagatgaacaaaaactctgttccaccagtgttctcatgctaaaaactttctgaaataccctaccctgttcaccgaatttctccacctgttggatagcagcattttactggtcgctgatggtcacttcaacctccacacctggctcaatggtgattgaggtgatctgcttgacaacatctgcagagctgacaagatcaatcacccttttgtgcacccgcatctcaaaccgatcccaggtgttggtacctgaaacaatgtttacatcattaacatttccaaccgcccatgggagatgcagaacaaagagggcaa

SEQ ID NO: 9 - DNA, 6270 bp, synthetic construct
  source 1..6270: mol_type = other DNA
cactaagccaacatagatatattaggatgcattgatcctggctgctgccacattcaaaagaccaagagcagcacgccatcttgaatttgactctttggttcatgctgggtagcctttcaaagccccaaaagattgggtcgggtctctctttcggctctcgtcggtggctgcttcccacaaagaccgccatcagacgtgaatgaactgcaagtctgcaactacgactccagttgctatccccttaaattactttactactacctttattctaagggccggttcggataattgccaaaatcaatcgtaccaaattttaataatattaggatgaaataaatatgttgcgccaaaattttctactgttactgaaatttggtaacaaactaaacacaaccgtatatttatcaattttaccaaatagtggtatggtttaaaatgacattaatctaaataggtcctgaaatataaaaatgcaccaaccatcaatgcatgcagttacattccaaagttctcctaggacacatatgccatatattttcgatggatagccttaatgtctgactactctcttcatcgcaacaaatctaatataatacaaatctgaataggagcttatataaatatatagtagtagaatatgtcataatattacagtggtattccatattggatttgttgattatagaacgacgaaagtatgcgttaacttacaaggttgtttacattatgacgaaatgaagagaaggaacgcacctactgcgaagagctcaacaactacttcctccaaatttccactcaccaactgatcaccaccaattcacttgtgcagtgccacctacccaatttcattccagtggcgtggtacgtaggttccgcatgtcagtgtgacaatcgtacggactgccatgtgtattgtgttcaggtaggtgcactggtcgtggtctcaccatctctcctccagtcgtacggcgccacgtaggcagtacacgtagacggccttcttcctcctccctcgagttgagttggttggtgagagtgagacaccgacggaacggaaggagaaccacgccgcttggatttttcttttttaccttttcaaattttaatttaaaaaataaaaccattttaaaaacttatcttcaaatacaaatcttttaaaaacactaacacgtgacacacagcgggcacgtcacccaaacgggcgtgacaatattgttttgccacaccaatccagctggtgtggacaaaatgttcatatattgaaaataaaatttaaaacaatttatattttttatctatatcattataaaaattgaagatgtttttaccggtattttgttactcatttgtgcatgagtcggtttttaagtttgttcgcttttggaaatacatatccgtatttgagtatgtttttaagttcgttcgttttttgaaatacaaaaggaatcgtaaaataaatctattttaaaaaactcgcatgctaacttgagacgatcgaactgctaattgcagctcataattttccaaaaaaaaatatatccaaacgagttcttatagtagatttcaccttaattaaaacatataaatgttcacccggtacaacgcacgagtatttttataagtaaaattaaaagtttaaaataaataaaaatcccgccaccacggcgcgatggtaaaagggggacgcttctaaacgggccgggcacgggacgatcggccccgaacccggcccatctaaccgctgtaggcccaccgcccaccaatccaactccgtactacgtgaagcgctggatccgcaacccgttaagcagtccacacgactcgactcgactcgcgcactcgccgtggtaggtggcaacccttcttcctcctctatttcttcttcttcctcccttctccgcctcaccacaccaaccgcaccaaccccaaccccgcgcgcgctctcccctctcccctcccaccaaccccaccccatcctcccgacctccacgccgccggcaatggcggcgaccatggcgtccaacgccgcggctgcggcggcggtgtccctggaccaggccgtggcggcgtcggcggcgttctcgtcgcggaagcagctgcggctgcccgccgcggcgcgcggggggatgcgggtgcgggtgcgggcgcgggggcggcgggaggcggtggtggtggcgtccgcgtcgtcgtcgtcggtggcagcgccggcggcgaaggcggaggagatcgtgctccagcccatcagggagatctccggggcggttcagctgccagggtccaagtcgctctccaacaggatcctcctcctctccgccctctccgaggtgagacgcggatcccttcctcttgcgtgaattccatttctggagatgagattttagggggtttattaggtgaggtggctgtgtttgtgaaatcctaggaattatctctcaagtcaatctaacgatgagatataactgaggttctggttttaatcacacactcatataaccaatttattgaaacattttggtttggcataagaaactgcttacgaaggtatgatatcctcctacatgtcaggctactaaattttcacgacggtatgatccactcaaaacaagtttcttaacgagtctggtgaggtctgttatgaaatttgtgtaaactaaggcaactttggaggtttcgcactgtaccaatgttatgtttgaacattttgcaagcagtgctttctcccaaaattatgcaattttgaggctcctctacatcattataattccccaatacattgctctttattcttaatagctttgatcgcgaaatttaacattttaattcttgagctgttattttgtagcatcagtttatcatgagccatgtttggtactaaatatacaatcccttgggtttatttgtttccaagcatgtcattaacttatcttaatgtggacaagaaactgatgcctgcttacattgctattatttcaagcgggtattgatcctttgacatgtgattgatcatttttttttctctggttattagggcacaacagtggtggacaacttgctgaacagtgaggatgttcactacatgcttgaggccctgaaagccctcgggctctctgtggaagcagataaagttgcaaaaagagctgtagtcgttggctgtggtggcaagtttcctgttgagaaggatgcgaaagaggaagtgcaactcttcttggggaacgctggaactgcaatgcgaccattgacagcagccgtgactgctgctggtggaaatgcaacgtatgttttttttttttttaatgtttatgaaaatatgtatggaattcatggggtatgttttatgacctttttctttaccatcagttatgtgcttgatggagtgccacgaatgagggagagaccgattggtgacttggttgtcgggttgaaacaacttggtgcggatgtcgactgtttccttggcactgaatgcccacctgttcgtgtcaagggaattggaggacttcctggtggcaaggttagttactcctaaactgcatcctttgtacttctgtatgcacctcaattctttgtcaaccttctgcatttataaggaacattctatgatgcaattcgaccttacactgcacagtaacttgaaatgtttcatgcttaatcaatatgccatattcctgccaagctcaagcgagcaatatttgtttgaatttggtaccatatttttgtatatttgggcattcctttttggtcttgatgtcttcttttgaattagcatttaactgaattacactcaacaggttaagctctctggttccatcagcagtcagtacttgagtgccttgctgatggctgctcctttggcccttggggatgtggagatcgaaatcattgacaaactaatctccattccttacgttgaaatgacattgagattgatggagcgttttggtgtgaaggcagagcattctgatagttgggacagattctatattaagggagggcagaagtacaagtaagcttctacctgccttactgagctgaattattcgggtgtttatgattaactccctaaactaaccctttttcttttctttggcattgacagatctcctggaaatgcctatgttgaaggtgatgcctcaagcgcgagctatttcttggctggtgctgcaatcactggaggcactgtgacagttcaaggttgtggtacgaccagtttgcaggtataactgtagtgcctgttttgacattctaccgtttagtcaagtttagtcagtagtcacatattcagaatatagcacaatctgtattatgccactgttaatcaaatacgcttgacctagagagtgctatataccctagcttaatcttcaaactaaacagttctcttgtggcttgctgtgctgttatgttccctgacctacatgttaatattacagggtgatgtcaaatttgctgaggtacttgagatgatgggagcaaaggttacatggactgacaccagtgtaaccgtaactggtccaccacgtgagccttatgggaagaaacacctgaaagctgttgatgtcaacatgaacaaaatgcctgatgttgccatgacccttgccgttgttgcactcttcgctgatggtccaactgctatcagagatggtaaacattaaggcctattatacctgttctatcatactagcaattactgcttagcattgtgacaaaacaaataaccaaactttcttcaaaataacttagaaatataagaaaggttcgttttgtgtggtaaacagtactactgtagtttcagctatgaagtttgctgctggcaattttctgaacggtttcagctaaattgcatgtttgttcatcatacttatccattgtcttccacagtggcttcctggagagtaaaggaaaccgaaaggatggttgcaattcggaccgagctaacaaaggtaaattcattaggtcccgtgtcctttcattcttcaagtagtttgttcataagttgaattctccttcaatgatgtttaaattcatcatcttcttttttggtgttgtgccagctgggagcatcggttgaagaaggtcctgactactgcatcatcaccccaccggagaagctgaacatcacggcaatcgacacctacgatgatcacaggatggccatggccttctccctcgctgcctgcgccgacgtgcccgtgacgatcagggaccctggttgcacccgcaagaccttccccaactacttcgacgttctaagcactttcgtcaggaactgaactgagcttttaaaagagtgaggtctaggttctgttgtctgtctgtccatcatccatgtgttgactgttgagggaactcgtttcttcttttcttcacgagatgagtttttgtgtgcctgtaatactagtttgtagcaaaggctgcgttacataaggtgatgagaattgaggtaaaatgagatctgtacactaaattcattcagactgttttggcataaagaataatttggccttctgcgatttcagaagctataaattgccatctcactaaattctccttggtcctcatggcaatgcaacgacagtgtgaagcactgaagcccgtaatgctctatcaccaccatgtacaacagaaccatatatgtccatatgtacaactcgagtgttgtttgagtggccagcaaactggctgaccaagccacacgagagagaatactataaactcaatcatacataacaagcccaagcaacagaagacagaacacaacaacactcgagtctgggaacagcaacgccgatacactacagcatactgcaacgacacaaaggaattggtaaggaaatggcaccaaaatcttgtagatctttagcgagtcattcatttatagcatatgttggaacagacatgagtcacaagatttatgatagcttagataacagatggtcggaattaaccgccgagagcctatagatgaacaaaaactctgttccaccagtgttctcatgctaaaaactttctgaaataccctaccctgttcaccgaatttctccacctgttggatagcagcattttactggtcgctgatggtcacttcaacctccacacctggctcaatggtgattgaggtgatctgcttgacaacatctgcagagctgacaagatcaatcacccttttgtgcacccgcatctcaaaccgatcccaggtgttggtacctgaaacaatgtttacatcattaacatttccaaccgcccatgggagatgcagaacaaagagggcaa

SEQ ID NO: 10 - DNA, 324 bp, synthetic construct
  source 1..324: mol_type = other DNA
atggcggcgaccatggcgtccaacgccgcggctgcggcggcggtgtccctggaccaggccgtggcggcgtcggcggcgttctcgtcgcggaagcagctgcggctgcccgccgcggcgcgcggggggatgcgggtgcgggtgcgggcgcgggggcggcgggaggcggtggtggtggcgtccgcgtcgtcgtcgtcggtggcagcgccggcggcgaaggcggaggagatcgtgctccagcccatcagggagatctccggggcggttcagctgccagggtccaagtcgctctccaacaggatcctcctcctctccgccctctccgag

SEQ ID NO: 11 - DNA, 704 bp, synthetic construct
  source 1..704: mol_type = other DNA
gtgagacgcggatcccttcctcttgcgtgaattccatttctggagatgagattttagggggtttattaggtgaggtggctgtgtttgtgaaatcctaggaattatctctcaagtcaatctaacgatgagatataactgaggttctggttttaatcacacactcatataaccaatttattgaaacattttggtttggcataagaaactgcttacgaaggtatgatatcctcctacatgtcaggctactaaattttcacgacggtatgatccactcaaaacaagtttcttaacgagtctggtgaggtctgttatgaaatttgtgtaaactaaggcaactttggaggtttcgcactgtaccaatgttatgtttgaacattttgcaagcagtgctttctcccaaaattatgcaattttgaggctcctctacatcattataattccccaatacattgctctttattcttaatagctttgatcgcgaaatttaacattttaattcttgagctgttattttgtagcatcagtttatcatgagccatgtttggtactaaatatacaatcccttgggtttatttgtttccaagcatgtcattaacttatcttaatgtggacaagaaactgatgcctgcttacattgctattatttcaagcgggtattgatcctttgacatgtgattgatcatttttttttctctggttattag

SEQ ID NO: 12 - DNA, 245 bp, synthetic construct
  source 1..245: mol_type = other DNA
ggcacaacagtggtggacaacttgctgaacagtgaggatgttcactacatgcttgaggccctgaaaggcctcgggctctctgtggaagcagataaagttgcaaaaagagctgtagtcgttggctgtggtggcaagtttcctgttgagaaggatgcgaaagaggaagtgcaactcttcttggggaacgctgcgactgcaatgcgatccttgacagcagccgtgactgctgctggtggaaatgcaac

SEQ ID NO: 13 - DNA, 84 bp, synthetic construct
  source 1..84: mol_type = other DNA
gtatgttttttttttttttaatgtttatgaaaatatgtatggaattcatggggtatgttttatgacctttttctttaccatcag

SEQ ID NO: 14 - DNA, 154 bp, synthetic construct
  source 1..154: mol_type = other DNA
ttatgtgcttgatggagtgccacgaatgagggagagaccgattggtgacttggttgtcgggttgaaacaacttggtgcggatgtcgactgtttccttggcactgaatgcccacctgttcgtgtcaagggaattggaggacttcctggtggcaag

SEQ ID NO: 15 - DNA, 277 bp, synthetic construct
  source 1..277: mol_type = other DNA
gttagttactcctaaactgcatcctttgtacttctgtatgcacctcaattctttgtcaaccttctgcatttataaggaacattctatgatgcaattcgaccttacactgcacagtaacttgaaatgtttcatgcttaatcaatatgccatattcctgccaagctcaagcgagcaatatttgtttgaatttggtaccatatttttgtatatttgggcattcctttttggtcttgatgtcttcttttgaattagcatttaactgaattacactcaacag

SEQ ID NO: 16 - DNA, 215 bp, synthetic construct
  source 1..215: mol_type = other DNA
gttaagctctctggttccatcagcagtcagtacttgagtgccttgctgatggctgctcctttggcccttggggatgtggagatcgaaatcattgacagactaatctccattccttacgttgaaatgacattgagattgatggagcgttttggtgtgaaggcagagcattctgatagttgggacagattctatattaagggagggcagaagtacga

SEQ ID NO: 17 - DNA, 93 bp, synthetic construct
  source 1..93: mol_type = other DNA
gtaagcttctacctgccttactgagctgaattattcgggtgtttatgattaactccctaaactaaccctttttcttttctttggcattgacag

SEQ ID NO: 18 - DNA, 118 bp, synthetic construct
  source 1..118: mol_type = other DNA
atctcctggaaatgcctatgttgaaggtgatgcctcaagcgcgagctatttcttggctggtgctgcaatcactggaggcactgtgacagttcaaggttgtggtacgaccagtttgcag

SEQ ID NO: 19 - DNA, 216 bp, synthetic construct
  source 1..216: mol_type = other DNA
gtataactgtagtgcctgttttgacattctaccgtttagtcaagtttagtcagtagtcacatattcagaatatagcacaatctgtattatgccactgttaatcaaatacgcttgacctagagagtgctatataccctagcttaatcttcaaactaaacagttctcttgtggcttgctgtgctgttatgttccctgacctacatgttaatattacag

SEQ ID NO: 20 - DNA, 211 bp, synthetic construct
  source 1..211: mol_type = other DNA
ggtgatgtcaaatttgctgaggtacttgagatgatgggagcaaaggttacatggactgacaccagtgtaaccgtaactggtccaccacgtgagccttatgggaagaaacacctgaaagctgttgatgtcaacatgaacaaaatgcctgatgctgccatgacccttgccgttgttgcactcttcgctgatggtccaactgctatcagagatg

SEQ ID NO: 21 - DNA, 237 bp, synthetic construct
  source 1..237: mol_type = other DNA
gtaaacattaaggcctattatacctgttctatcatactagcaattactgcttagcattgtgacaaaacaaataaccaaactttcttcaaaataacttagaaatataagaaaggttcgttttgtgtggtaaacagtactactgtagtttcagctatgaagtttgctgctggcaattttctgaacggtttcagctaaattgcatgtttgttcatcatacttatccattgtcttccacag

SEQ ID NO: 22 - DNA, 62 bp, synthetic construct
  source 1..62: mol_type = other DNA
tggcttcctggagagtaaaggaaaccgaaaggatggttgcaattcggaccgagctaacaaag

SEQ ID NO: 23 - DNA, 111 bp, synthetic construct
  source 1..111: mol_type = other DNA
gtaaattcattaggtcccgtgtcctttcattcttcaagtagtttgttcataagttgaattctccttcaatgatgtttaaattcatcatcttcttttttggtgttgtgccag

SEQ ID NO: 24 - DNA, 219 bp, synthetic construct
  source 1..219: mol_type = other DNA
ctgggagcatcggttgaagaaggtcctgactactgcatcatcaccccaccggagaagctgaacatcacggcaatcgacacctacgatgatcacaggatggccatggccttctccctcgctgcctgcgccgacgtgcccgtgacgatcagggaccctggttgcacccgcaagaccttccccaactacttcgacgttctaagcactttcgtcaggaactga

SEQ ID NO: 25 - DNA, 27 bp, synthetic construct
  source 1..27: mol_type = other DNA
caagcagtgctttctcccaaaattatg

SEQ ID NO: 26 - DNA, 30 bp, synthetic construct
  source 1..30: mol_type = other DNA
aatgctaattcaaaagaagacatcaagacc

SEQ ID NO: 27 - DNA, 27 bp, synthetic construct
  source 1..27: mol_type = other DNA
ggaaatgcttatgttgaaggagatgca

SEQ ID NO: 28 - DNA, 30 bp, synthetic construct
  source 1..30: mol_type = other DNA
ttcgaagatctagtaacatagatgacaccg